(19) United States
(12) Patent Application Publication
(10) Pub. No.: US 2020/0384150 A1
(43) Pub. Date: Dec. 10, 2020

(54) BIOCAMPATIBLE HEAT AND y-RADIATION STABLE MEDICAL DEVICE LUBRICANT AND CORROSION PREVENTATIVE

(71) Applicant: Depuy Synthes Products, Inc., Raynham, MA (US)

(72) Inventor: Allan KIMBLE, West Chester, PA (US)

(21) Appl. No.: 16/431,899

(22) Filed: Jun. 5, 2019

(51) Int. Cl. A61L 27/14; A61L 27/04; A61L 31/02; A61L 31/16; A61L 27/54

(52) U.S. Cl. CPC A61L 27/14; A61L 27/04; A61L 29/04; A61L 31/16; A61L 27/54; A61L 31/022

(57) ABSTRACT

A metal surgical instrument having improved corrosion resistance, wherein the surgical instrument is treated with a biocompatible polyphenyl ether-based polymeric coating.

### TECHNICAL FIELD

Various exemplary embodiments disclosed herein relate to the use of polyphenyl ether (PPE)-based coatings as non-toxic, thermal and radiation stable lubricants and corrosion preventatives for medical devices.

### BACKGROUND

Stainless steel and related alloys vary in corrosion resistance based upon their composition and exposure to various environments. Certain medical device alloys require the application of a preservative film to prevent corrosion. Water-based lubricant preservatives are unsuitable for some medical device materials, in particular, articulating instruments that are less corrosion resistant. Straight-chain hydrocarbons and silicone-based lubricants suffer from radiation damage that can render them less effective or toxic. Polytetrafluoroethylene (PTFE) based lubricants cannot withstand gamma radiation at typical sterilizing doses (20-25 kGy) without significant degradation.

Polyphenyl ethers (PPEs) were developed in the mid-1900's to meet the needs of the aerospace and nuclear industries for lubricants and hydraulic fluids that would withstand wide temperature ranges and exposure to high levels of radiation without changes in their properties or chemistry. An example of their early use was in exotic aircraft, satellites and nuclear reactor mechanical applications. Their main use today is as a lubricant for electrical connectors and as a minor additive in high performance lubricants.

### SUMMARY

A brief summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments disclosed herein relate to a surgical instrument including a metal surface configured to contact a surgical patient, and a biocompatible polymeric coating over the metal surface, wherein the polymeric coating includes a polyphenyl ether polymer, and wherein the coating slows the rate of corrosion of the metal surface during storage.

Various embodiments disclosed herein relate to a method of treating a metal surface of a surgical instrument to improve corrosion resistance, wherein the method includes coating the metal surface with a biocompatible polymeric coating, wherein the coating includes a polyphenyl ether polymer.

In various embodiments, the polyphenyl ether polymer is a five-ring polyphenyl ether.

In various embodiments, the metal is a stainless steel or molybdenum alloy.

In various embodiments, the coating is resistant to degradation by gamma radiation.

In various embodiments, the medical device is either sterile or non-sterile.

### BRIEF DESCRIPTION OF THE DRAWINGS

In order to better understand various exemplary embodiments, reference is made to the accompanying drawings, wherein:

FIG. 1 shows the DATR Spectra of Gamma-Sterilized (43-58 kGy) SANTOLUBE® OS-124 Coated Fluted Drums (Anspach Packaged).

FIG. 2 shows the DATR Spectra of non-sterile SANTOLUBE® OS-124 Coated Fluted Drums (Millstone Packaged).

FIG. 3 shows the preservation of a non-sterile SANTOLUBE® OS-124 Coated Fluted Drum, 4 weeks post-manufacturing.

FIG. 4 shows the preservation of a sterile SANTOLUBE® OS-124 Coated Fluted Drum, 4 weeks post-manufacturing.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art, and are to be construed as being without limitation to such specifically recited examples and conditions. Additionally, the term, “or,” as used herein, refers to a non-exclusive or (i e , and/or), unless otherwise indicated (e.g., “or else” or “or in the alternative”). Also, the various embodiments described herein are not necessarily mutually exclusive, as some embodiments can be combined with one or more other embodiments to form new embodiment.

The present disclosure provides for the use of polyphenyl ether (PPE)-based polymeric coatings as non-toxic, radiation stable lubricants and corrosion preventatives for medical devices, such as metal surgical instruments configured to contact surgical subjects. The PPE-based polymeric coatings are formulated to be resistant to degradation by gamma radiation or heat and provides a conformal barrier to corrosion. The PPE-based coatings of the present disclosure are biocompatible, can withstand both steam and gamma radiation sterilization and retain biocompatibility following sterilization. The PPE-based coatings further exhibit excellent resistance to oxidation, including oxidative resistance above 100° C.

The present disclosure further provides for surgical instruments which contain at least one metal surface and a biocompatible PPE-based polymeric corrosion resistant coating coated thereon, wherein the coating slows the rate of corrosion during the storage life of the instrument.

As used herein a “polymeric” material is one that contains one or more types of polymers, commonly containing at least 50 wt % to 75 wt %, to 90 wt % to 95 wt % to 99 wt % or even more polymers. Thus, polymeric materials include those containing a single type of polymer as well as polymer blends.

As used herein, “polymers” are molecules that contain multiple copies of one or more constitutional units, commonly referred to as monomers, and typically contain from 5 to 10 to 25 to 50 to 100 to 500 to 1000 or more constitutional units. Polymers may be, for example, homopolymers, which contain multiple copies of a single constitutional unit, or copolymers, which contain multiple copies of at least two dissimilar constitutional units, which units may be present in any of a variety of distributions including random, statistical, gradient, and periodic (e.g., alternating) distributions.

The PPE-based polymeric coating of the present disclosure may be used to lubricate surgical instruments or other sterile and non-sterile medical devices. Suitable PPE polymers include six-ring polyphenyl ether polymers, five-ring polyphenyl ether polymers, four-ring polyphenyl ether polymers, three- and four-ring oxy- and thioether polymers, three-ring polyphenyl ether polymers, two-ring diphenyl ether polymers, and combinations thereof. The PPE-based polymeric coating preferably contains a five-ring polyphenyl ether polymer.

The PPE-based coating may also contain other polymeric materials selected from: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-alkylene copolymers, including styrene-butadiene copolymers, styrene-ethylene-butylene copolymers, styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers, polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including other polyarylethers such as polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; as well as further copolymers of the above.

Preferred polymeric materials of the present disclosure are biocompatible and non-cytotoxic as determined using the MEM Elution subjective scoring method (Grade 0-4), wherein a passing score is Grade 0 or Grade 1. Other tests for biocompatibility and cytotoxicity may also be used and are known to those skilled in the art.

Preferred polymeric materials also provide corrosion resistance to the coated surgical instrument for at least 4 weeks upon storage of the instrument.

Preferred polymeric materials further provide gamma-radiation resistance to at least a level of 20 kGy, preferably 40-50 kGy, to the coated surgical instrument so as to prevent degradation of the PPE-based coating that would adversely affect corrosion resistance or biocompatibility of the instrument.

Specific medical devices that may be lubricated using the PPE-based coating include any surgical instruments having a metal surface. Exemplary surgical instruments include surgical scissors and other cutting instruments, electrosurgical instruments, cautery instruments, needle holders, osteotomes and periosteotomes, chisels, gouges, rasps, files, saws, reamers, wire twisting forceps, wire cutting forceps, ring handled forceps, tissue forceps, cardiovascular clamps, rongeurs, or any other hard metal instrument having teeth, serrations, a cutting edge or being otherwise susceptible to corrosion.

Specific hard metals that may be coated with the PPE-based coating of the present disclosure include steel-based materials including stainless steel, titanium or titanium alloy, iron-nickel alloy and molybdenum or molybdenum alloy or combinations thereof. Preferred hard metals include stainless steel alloy 15-SPH, 17-SPH, 300 series, 400 series; and M series steel molybdenum alloys or combinations thereof. Specific examples include high carbon martensitic stainless steel (e.g. Stainless steel Type 440A), high speed tool stainless steels containing molybdenum and tungsten, but not cobalt (e.g. M1 and M2), and high speed tool stainless steel alloys containing molybdenum, chromium, tungsten and vanadium (e.g. M7).

In other embodiments, medical devices that may be lubricated using the PPE-based coating include articulating implantable devices and instruments, including, but not limited to, distractors and adjustable spacers, and softer metallic devices, such as bending templates.

In some embodiments of the disclosure, the polymeric coatings are configured to result in reduced corrosion in certain areas of the instruments relative to other areas. For this purpose, some areas of the instrument may be provided with coating materials, while others are not. Moreover, coating materials may be provided which provide differing corrosion protection, for example due to a difference in the composition of the material making up the coating, due to a difference in the thickness of the coating material, and so forth. In this regard, the coating composition and/or thickness may change abruptly (e.g., in a stepwise fashion) and/or gradually along the surface of the instrument.

The PPE-based polymeric coating of the disclosed embodiments may be formed using any of a variety of techniques depending upon the polymer or polymers making up the coatings, including, for example, physical vapor deposition, chemical vapor deposition, electrochemical deposition, layer-by-layer techniques, and coating techniques based on the application of liquid polymer compositions, examples of which include polymer melts, polymer solutions, and curable polymer systems, among other techniques. In certain embodiments, the polymeric coating may be formed using dip-coating, spray-coating, web-coating or spin coating.

In exemplary embodiments, the coating may be applied before or after sterilization of the coated instrument and may prevent packaging material from sticking to the instrument.

### EXAMPLES

The specific polyphenyl ether preservative utilized in the Examples was SANTOLUBE® OS-124 High-Temperature Radiation-resistant Base Fluid, which is a 5-ring polyphenyl ether with exceptionally low volatility and resistance to degradation from heat, oxygen, radiation and chemical attack.

The hard metal alloy tested in the Examples is M2 steel which contains tungsten and does not contain cobalt.

The following materials were used in the Examples:

Polyphenyl Ether 30 mL in each of three (3) TOC vials

65-BLA-11-1 12.7 mm Fluted Drum 20 each.

The application of the polyphenyl ether is as directed in DMR Operating Procedure G01.05.01 Manual Cleaning of Cutters with the exception that instead of the HL-1640-00 oil, the polyphenyl ether SANTOLUBE® OS-124 High-temperature Radiation-resistant Base Fluid is used.

### Example 1

Cytotoxicity Evaluation

The Cytotoxicity Evaluation test matrices are shown below:

TABLE 1
Non-Sterilized Preserved Alloys

| Alloy | Polyphenyl ether |
|---|---|
| M2 | 1, 2, 3 |

TABLE 2
Gamma-Sterilized Preserved Alloys (40-50 kGy)

| Alloy | Polyphenyl ether |
|---|---|
| M2 | 4, 5, 6 |

Cytotoxicity was determined using the MEM Elution subjective scoring method (Grade 0-4) with results reported at 24, 48, and 72 hours post-24-hour extraction. A passing score is Grade 0, Grade 1 or Grade 2. Results of the cytotoxicity study with the above test samples packaged in different packaging are shown in Table 3:

TABLE 3
Cytotoxicity Results

| | | Sample 1 | | | Sample 2 | | | Sample 3 | | |
|---|---|---|---|---|---|---|---|---|---|---|
| Sample | Packaged | 24-Hr | 48-Hr | 72-Hr | 24-Hr | 48-Hr | 72-Hr | 24-Hr | 48-Hr | 72-Hr |
| Non-Sterile | Anspach | 0/0/0 | 0/0/0 | 0/0/0 | 0/0/0 | 1/1/1 | 1/1/1 | 0/0/0 | 0/0/0 | 0/0/0 |
| Sterile | Anspach | 0/0/0 | 0/0/0 | 0/0/0 | 0/0/0 | 1/1/1 | 1/1/1 | 0/0/0 | 0/0/0 | 1/1/1 |
| Non-Sterile | Millstone | 0/0/0 | 0/0/0 | 0/0/0 | 0/0/0 | 0/0/0 | 0/0/0 | 0/0/0 | 0/0/0 | 0/0/0 |

### Example 2

Intracutaneous Irritation Test

An intracutaneous irritation test was conducted to determine if PPE would leach or be extracted from a test sample and cause local irritation in the dermal tissues of albino rabbits. The test sample was diluted at a 1:1 ratio using sesame oil. Each animal was weighed and the weight recorded prior to test injection. The fur of the animals was clipped on both sides of the spinal column to expose a sufficient sized area for injection.

The test article and a vehicle control were injected into three rabbits. Each rabbit received five sequential 0.2 mL intracutaneous injections of the test article extract on the right side of the vertebral column and similarly the control vehicle on the left side.

The animals were observed daily for abnormal clinical signs. The appearance of each injection site was noted immediately post injection and at 24±2, 48±2 and 72±2 hours. The tissue reactions were rated for gross evidence of erythema and edema.

None of the animals on study showed abnormal clinical signs during the 24, 48 and 72 hour observation periods. There were no significant dermal reactions observed at the injected test and control sites on the rabbits at the 24, 48 and 72 hour observation periods.

### Example 3

Guinea Pig Maximization Sensitization Test

A test was conducted to evaluate the allergenic potential or sensitizing capacity of a test article containing PPE. The test was used as a procedure for the screening of contact allergens in guinea pigs and extrapolating the results to humans.

Eleven test guinea pigs were injected with the test article and Freund's Complete Adjuvant (FCA), and six control guinea pigs were injected with a control and FCA. On Day 6, the dorsal site was re-shaved and sodium lauryl sulfate (SLS) in mineral oil was applied. One week after the injections, the test animals were topically patched with the test article and the control animals were patched with the control. The patches were removed after 48±2 hours of exposure. Following an approximate two-week rest period, all animals were topically patched in a previously untreated area with the test article on the right fur-clipped right flank or dorsum as well as with the control on the fur-clipped left flank or dorsum. The patches were removed after 24±2 hours of exposure. The dermal patch sites were observed for erythema and edema 24±2 and 48±2 hours after patch removal. Each animal was assessed for a sensitization response based upon the dermal scores. The test results were based upon the percentage of animals exhibiting a sensitization response.

None of the animals in the study showed abnormal clinical signs during the test period. Additionally, none of the control animals challenged with the control solution were observed with a sensitization response greater than 0. None of the test animals challenged with the test article were observed with a sensitization response greater than 0. Accordingly, the test article did not elicit a sensitization response.

### Example 4

Acute Systemic Injection Test

An acute systemic injection test was conducted to screen test article solutions containing PPE for potential toxic effects as a result of a single-dose systemic injection in mice.

Animals were treated by the intraperitoneal route to screen the test article solutions for potential toxic effects as a result of a single-dose systemic injection. For the safety evaluation, mice were injected systemically with the test article solution or control sesame oil (SO). The animals were observed for signs of toxicity immediately after injection and at 4, 24, 48 and 72 hours post-injection. The requirements of the test were met if none of the animals treated with the test article had a significantly greater adverse reaction than the animals treated with a vehicle control.

None of the animals on study were observed with abnormal clinical signs indicative of toxicity during the 72 hour test period. All were alive at the end of the 72 hour test duration and body weight changes were within acceptable parameters over the course of the study. The vehicle control treated animals had no signs of toxicity at any of the observation periods and no animals lost weight in excess of 10% indicating a valid test. None of the test article treated animals were observed with clinical signs consistent with toxicity at any of the observation periods. Body weight changes were within acceptable parameters over the course of the study.

### Example 5

Qualitative Molecular Composition Evaluation (FTIR)

TABLE 3
Non-Sterilized Preserved Alloys

| Alloy | Polyphenyl ether |
|---|---|
| M2 | 7, 8, 9 |

TABLE 4
Gamma-Sterilized Preserved Alloys (40-50 kGy)

| Alloy | Polyphenyl ether |
|---|---|
| M2 | 10, 11, 12 |

Molecular stability was determined by observing for changes in the infrared spectra of the neat, non-sterilized and sterilized samples using FTIR-ATR methods. For the neat samples, the PPE preservative was applied to the internal reflecting element (IRE) directly and the spectrum measured in absorbance mode between 4000 cm⁻¹ and 600 cm⁻¹ in 1 cm⁻¹ increments and 64 co-added scans. For the test samples, the butterfly bar was pressed against the IRE and the spectrum obtained using the same wave number range as for the neat sample.

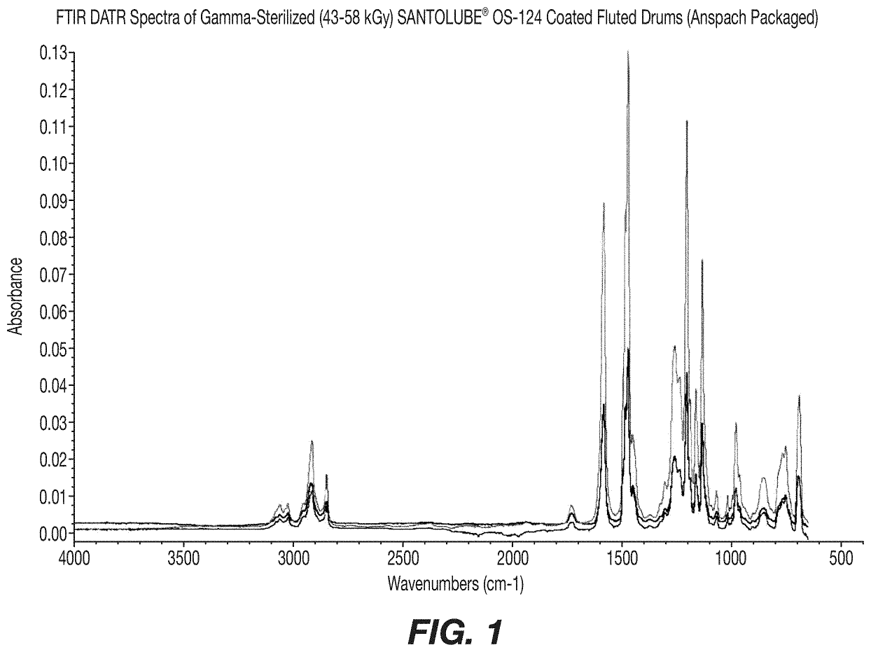

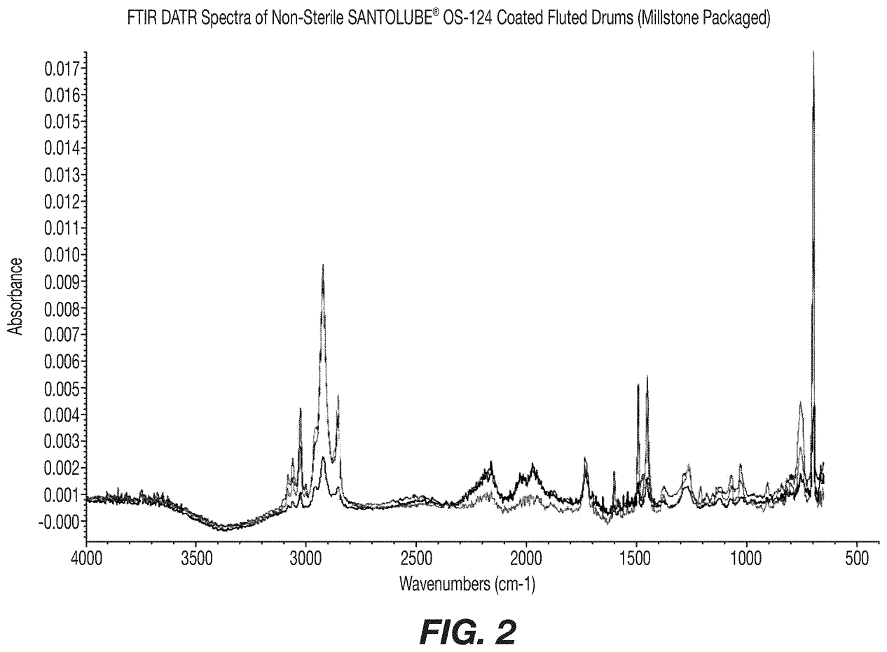

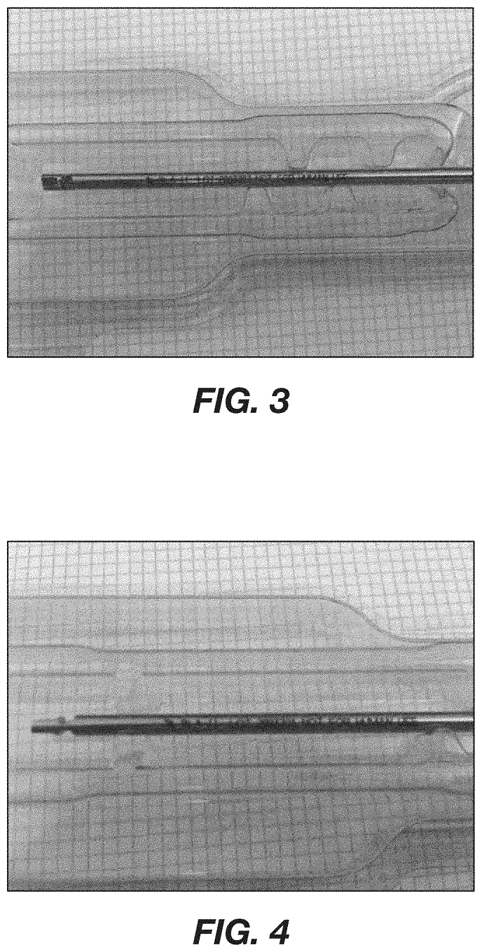

Results of the stability test are shown in FIGS. 1 and 2.

### Example 6

Corrosion Preservation Evaluation

After establishing that the preservative in the non-sterile and gamma sterilized states was non-cytotoxic, the corrosion preservative properties of PPE was evaluated.

TABLE 5
Non-sterilized Preserved Alloys

| Alloy | Polyphenyl ether |
|---|---|
| M2 | 15, 16, 17 |

TABLE 6
Gamma-Sterilized Preserved Alloys (40-50 kGy)

| Alloy | Polyphenyl ether |
|---|---|
| M2 | 18, 19, 20 |

TABLE 7
Non-Preserved Alloys

| Alloy | Polyphenyl ether |
|---|---|
| M2 | 13, 14 |

Each of the test devices listed in Tables 5-7 were placed into packaging material and stored under ambient conditions for two weeks (14 days) and then observed for the presence of visible corrosion. If corrosion was observed on the unpreserved samples, then the test was considered completed. Observation of the non-sterile and sterile test devices stored under the same conditions was performed and the presence of visible corrosion reported.

Additionally, samples that were cleaned, preserved and packaged by Millstone Medical Outsourcing was implemented as part of the overall evaluation. The samples were non-sterile samples.

Data for the cytotoxicity, molecular stability and corrosion evaluations are summarized in Table 8 below:

| | | | | | | Visible |
|---|---|---|---|---|---|---|
| | | | | MEM | Δ FTIR | Corrosion |
| Sample | Laboratory Sample | | | Cytotoxicity | Spectral | at 4 |
| ID | Code | Alloy | Sterile? | Score (0-4) | Results? | weeks? |
| 1 | 1-NS-P-1 | M2 | N | 0/0/0 | | |
| 2 | 1-NS-P-2 | M2 | N | 0/1/1 | | |
| 3 | 1-NS-P-3 | M2 | N | 0/0/0 | | |
| 4 | 2-S-P-1 | M2 | Y | 0/0/0 | | |
| 5 | 2-S-P-2 | M2 | Y | 0/1/1 | | |
| 6 | 2-S-P-3 | M2 | Y | 0/0/1 | | |
| 7 | 7 | M2 | N | | None by DATR | |
| 8 | 8 | M2 | N | | None by DATR | |
| 9 | 9 | M2 | N | | None by DATR | |
| 10 | 10 | M2 | Y | | None by DATR | |
| 11 | 11 | M2 | Y | | None by DATR | |
| 12 | 12 | M2 | Y | | None by DATR | |
| 13 | Unpreserved | M2 | N | | | N |
| 14 | Unpreserved | M2 | N | | | N |
| 15 | NA | M2 | N | | | N |
| 16 | NA | M2 | N | | | N |
| 17 | NA | M2 | N | | | N |
| 18 | NA | M2 | Y | | | N |
| 19 | NA | M2 | Y | | | N |
| 20 | NA | M2 | Y | | | N |
| 21M | 1-NS-P-M | M2 | N | 0/0/0 | | N |
| 22M | 2-NS-P-M | M2 | N | 0/0/0 | | N |
| 23M | 3-NS-P-M | M2 | N | 0/0/0 | | N |
| 24M | 24 | M2 | N | | None by DATR | |
| 25M | 25 | M2 | N | | None by DATR | |
| 26M | 26 | M2 | N | | None by DATR | |
| 27M | NA | M2 | N | | | N |
| 28M | NA | M2 | N | | | N |
| 29M | NA | M2 | N | | | N |

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be effected while remaining within the spirit and scope of the invention. Further, various elements from the various embodiments may be combined to form other embodiments that are within the spirit and scope of the invention. Accordingly, the foregoing disclosure, description, and figures are for illustrative purposes only and do not in any way limit the invention, which is defined only by the claims.

What is claimed is:

1. A surgical instrument comprising a metal surface configured to contact a surgical patient and a biocompatible polymeric coating over the metal surface, wherein the polymeric coating comprises a polyphenyl ether polymer, and wherein the coating slows the rate of corrosion of the metal surface during storage.

2. The surgical instrument of claim 1, wherein the polyphenyl ether polymer is selected from the group consisting of a six-ring polyphenyl ether polymer, a five-ring polyphenyl ether polymer, a four-ring polyphenyl ether polymer, a three- and four-ring oxy- and thioether polymer, a three-ring polyphenyl ether polymer, a two-ring diphenyl ether polymer, or a combination thereof.

3. The surgical instrument of claim 2, wherein the polyphenyl ether polymer is a five-ring polyphenyl ether polymer.

4. The surgical instrument of claim 1, wherein the metal surface comprises metal alloys selected from the group consisting of stainless steel, titanium or titanium alloy, iron-nickel alloy, molybdenum alloy or combinations thereof.

5. The surgical instrument of claim 4, wherein the metal alloy is selected from the group consisting of a stainless steel alloy, a molybdenum alloy, or a combination thereof.

6. The surgical instrument of claim 5, wherein the metal alloy comprises an M2 molybdenum alloy.

7. The surgical instrument of claim 1, wherein the polymeric coating is resistant to degradation by gamma radiation.

8. The surgical instrument of claim 1, wherein the instrument is a cutting instrument or an articulating instrument.

9. The surgical instrument of claim 1, wherein the surgical instrument is either sterile or non-sterile.

10. The surgical instrument of claim 9, wherein the surgical instrument is sterile.

11. A method of treating a metal surface of a surgical instrument to improve corrosion resistance, wherein the method comprises coating the metal surface with a biocompatible polymeric coating, wherein the polymeric coating comprises a polyphenyl ether polymer.

12. The method of claim 11, wherein the polyphenyl ether polymer is selected from the group consisting of a six-ring polyphenyl ether polymer, a five-ring polyphenyl ether polymer, a four-ring polyphenyl ether polymer, a three- and four-ring oxy- and thioether polymer, a three-ring polyphony ether polymer, a two-ring diphenyl ether polymer, or a combination thereof.

13. The method of claim 12, wherein the polyphenyl ether polymer is a five-ring polyphenyl ether polymer.

14. The method of claim 11, wherein the metal surface comprises metal alloys selected from the group consisting of stainless steel, titanium or titanium alloy, iron-nickel alloy, molybdenum alloy or combinations thereof.

15. The method of claim 14, wherein the metal alloy is selected from the group consisting of stainless steel alloy, molybdenum alloy, or combinations thereof.

16. The method of claim 15, wherein the metal alloy comprises an M2 molybdenum alloy.

17. The method of claim 11, wherein the polymeric coating is resistant to degradation by gamma radiation.

18. The method of claim 11, wherein the surgical instrument is a cutting instrument or an articulating instrument.

19. The method of claim 11, wherein the surgical instrument is either sterile or non-sterile.

20. The method of claim 19, wherein the surgical instrument is sterile.
